(19) United States
(12) Patent Application Publication
(10) Pub. No.: US 2008/0188711 A1
(43) Pub. Date: Aug. 7, 2008

(54) DUAL-ACTION ROTATIONAL AND LINEAR ACTUATING MECHANISM

(76) Inventors: Eliahu ELIACHAR, Haifa (IL); Dan Sade Hochstadter, Kibbutz Bet Alfa (IL); Ofer Yossepowitch, Petach Tikva (IL); Nir Lilach, Kfar Yehoshua (IL); Ram Grossfeld, Haifa (IL)

Correspondence Address: Fleit Gibbons Gutman Bongini & Bianco PL, 21355 EAST DIXIE HIGHWAY, SUITE 115, MIAMI, FL, 33180, US

(21) Appl. No.: 12/049,473

(22) Filed: Mar. 17, 2008

Related U.S. Application Data
(63) Continuation of application No. PCT/IL2006/001024, filed on Sep. 4, 2006.
(60) Provisional application No. 60/716,927, filed on Sep. 15, 2005.

(51) Int. Cl. A61B 1/00; G05G 9/00

(52) U.S. Cl. 600/106; 74/471 R

(57) ABSTRACT

The present invention provides a dual action linear-rotational resectoscope comprising: (a) a resection loop and loop shaft which are raised to high electrical tension for either resection or cauterisation; (b) an endoscope tube electrically insulated from the loop shaft; (c) an insulating sheath comprising an inner and outer sheath between which fluid is free to flow, surrounding both the loop shaft and the endoscope tube; (d) a handle by which an operator controls both linear and rotational motion of the resector-loop such that the operator performs a resection by using either rotational motion of the resector-loop, linear motion of the resector-loop or any combination of the two; and, (e) a dual-action actuating mechanism useful for manoeuvring the resector-loop adjacent to a body portion to be treated in a sequence of rotational and linear maneuvers; the maneuvers are selected from a group consisting of linear motion followed by rotational motion, rotational motion followed by linear motion, only rotational motion and only linear motion; comprising a unified actuating module in communication with the effecter.

### CROSS REFERENCE TO RELATED APPLICATIONS

This application is a continuation of International Patent Application No. PCT/IL2006/001024 filed Sep. 4, 2006, which claimed priority to U.S. Provisional Patent Application No. 60/716,927 filed Sep. 15, 2005.

### FIELD OF THE INVENTION

The present invention generally relates to a dual-action rotational and linear actuating mechanism, and the same when applied in an endoscopic tool, preferably a resectoscope.

### BACKGROUND OF THE INVENTION

Resectoscopes are medical devices useful for the resection of biological tissue, usually in order to remove pathologies in the tissue or to sample suspect tissue. Resectoscopes are elongated, narrow devices, which penetrate mammalian cavities. Typically, the resectoscope's distal end is positioned in the cavity and its proximal end is located outside the body. Resectoscopes comprise inter alia an elongated optical system and an actuator, wherein the actuator has means to translate a movement of at least one handle along the longitudinal axis of the resectoscope to the movement of a resecting loop, wherein the loop is connected to an electrical source and thus has means to resect the desired tissue along the longitudinal axis.

All resectoscopes known in the art comprise a cutting member, wherein cutting is enabled by means of an electrical current, which produces sufficient heat to coagulate and cut tissue. The heat burns the resected tissues so further analysis of the tissue is impaired.

Most resectoscopes in clinical use act by employing a linear motion, whereby a forward and backward motion of a high frequency electrocautery loop performs the cutting through the tissues.

Nevertheless, US patent applications 20040064139 and 20050171531 to the inventors disclose resectoscopes that perform a similar function by using rotational motion, whereby circular motion of the loop performs the cut. Those newly defined endoscopic tools present an accurate working tool for resectoscopes for lateral, side to side resection of biological tissue, a resectoscope including such a working tool, and a method for using such a working tool. Cutting member assemblies and cutting members suitable for lateral resections are also provided in the present invention. In embodiments of the present invention, vibratory or oscillatory motion is applied by a means for vibration to the cutting member assembly as the assembly rotates

In both systems the surgeon controls a singular motion characteristic of the loop from a handset attached to the shaft of the resectoscope.

The working element for the linear motion resectoscope is distinct from the working element of the rotational motion resectoscope. When a surgeon mechanically activates the handset of a linear motion resectoscope the working element produces a linear motion which is transmitted directly to the loop. Alternately, when a surgeon mechanically activates the handset of a rotational motion resectoscope the handset produces a linear motion which is transmitted to the loop in a circular manner. It is not currently possible to combine rotational motion and linear motion of the loop in a single working element. Two separate handsets are required for linear and rotational motion of the loop.

Although the rotational resectoscope is believed to provide a better surgical result by allowing a more accurate resection, its major disadvantage becomes the inability to retract the loop into the resectoscope sheath when required. Consequently, abutting the tip of the optical lens to the tissue so as to enable better visualization of bleeding vessels is impossible.

### SUMMARY OF THE INVENTION

It is thus one object of the present invention to disclose a dual-action actuating system, interconnecting an effecter with a single actuating mechanism activating said effecter in a sequence of rotational and linear maneuvers.

It is in the scope of the present invention to disclose a working tool comprising the dual-action system as defined above. The tool is characterized by a main longitudinal axis having a distal end and a proximal end. At least one effecter is located in the distal end, being in a communication with the single actuating mechanism, located at the proximal portion of the tool. The single actuating mechanism is preferably accommodated at the proximal end, namely in or near the tool's handle.

It is also in the scope of the present invention to disclose a working tool as defined in any of the above, wherein the single actuating mechanism comprises inter alia at least one active handle. The handle is preferably located at the vicinity of the proximal end and provides the effecter with both a linear motion and a rotational motion. More specifically, the working tool defined above is comprised of an effecter and single actuating mechanism itself comprising a rotation mechanism. The rotational mechanism is selected in anon-limiting manner from rotating screw and fixed bolt, rotating bolt and fixed screw, a spiral track and travelling pin or any other means of producing rotational motion. The linear motion mechanism is selected in a non-limiting manner from a sliding lock plate which pushes the rotational mechanism in a linear direction, a straight track and travelling pin or any other means of providing linear motion, such that an operator uses the active handle to produce both rotational and linear motion of the communicated effecter.

It is also in the scope of the present invention to disclose a working tool as defined in any of the above, wherein the linear mechanism is bolted to a springed arm; the extension of the springed arm is impeded by an active handle, by which the operator controls the extent of the linear motion, during the predetermined portion of the full range of the handle. When fully extended, the linear motion springed arm prevents any further movement of the linear movement mechanism and further wherein said rotational motion mechanism is bolted to the active handle such that its movement is controlled directly by the operator over the second portion of the full range of the active handle. It is acknowledged in this respect that the aforesaid tool is alternatively characterized in the manner that the first motion is rotational and the second motion is linear. A tool with a multiple motions sequence is also possible, e.g., a liner-rotational-liner motion, rotational-linear-rotational motion etc.

It is a further object of the present invention to disclose an endoscope having a dual-action actuating mechanism, especially useful for manoeuvring at least one effecter located adjacent to a body portion to be treated in a sequence of rotational and linear maneuvers comprising a unified actuating module in communication with said effecter.

It is thus in the scope of the present invention to disclose the aforesaid endoscope comprising tool characterized by a main longitudinal axis, said main axis having a distal end and a proximal end. At least one effecter is located in the distal end, being in a communication with the single actuating mechanism, located at the proximal portion of the tool. The single actuating mechanism is preferably accommodated at the proximal end, namely in or near the tool's handle. The single actuating mechanism preferably comprises at least one active handle, located at the vicinity of the proximal end, and provides the effecter with both a linear motion and a rotational motion.

It is also in the scope of the present invention to disclose an endoscope as defined above, comprising an effecter and single actuating mechanism itself comprising a rotation mechanism. The rotation mechanism of the endoscope is selected in a non-limiting manner from rotating screw and fixed bolt, rotating bolt and fixed screw, a spiral track and travelling pin or any other means of producing rotational motion. The linear motion mechanism of the endoscope is selected in a non-limiting manner from a sliding lock plate which pushes the rotational mechanism in a linear direction, a straight track and travelling pin or any other means of providing linear motion, such that an operator uses the active handle to produce both rotational and linear motion of the communicated effecter.

It is also in the scope of the present invention to disclose an endoscope as defined above, wherein said linear mechanism is bolted to a springed arm. The extension of the springed arm is impeded by an active handle, by which the operator controls the extent of the linear motion, during the predetermined portion of the full range of the handle; when fully extended, the linear motion springed arm prevents any further movement of the linear movement mechanism and further wherein said rotational motion mechanism is bolted to the active handle such that its movement is controlled directly by the operator over the second portion of the full range of the active handle. It is acknowledged in this respect that the aforesaid endoscope is alternatively characterized in the manner that the first motion is rotational and the second motion is linear. An endoscope with a multiple motions sequence is also possible, e.g., a liner-rotational-liner motion, rotational-linear-rotational motion etc.

It is still another object of the present invention to present a dual action linear-rotational resectoscope. This novel resectoscope comprising inter alia (a) a resection loop and loop shaft which are raised to high electrical tension for either resection or cauterisation; (b) an endoscope tube electrically insulated from the loop shaft; (c) an insulating sheath comprising an inner and outer sheath between which fluid is free to flow, surrounding both the loop shaft and the endoscope tube; (d) a handle by which an operator controls both linear and rotational motion of the resector-loop such that the operator performs a resection by using either rotational motion of the resector-loop, linear motion of the resector-loop or any combination of the two; and (e) a dual-action actuating mechanism useful for manoeuvring said resector-loop adjacent to a body portion to be treated in a sequence of rotational and linear maneuvers comprising a unified actuating module in communication with said effecter.

It is in the scope of the present invention to disclose a resectoscope comprising the dual-action system as defined above. The resectoscope is preferably characterized by a main longitudinal axis having a distal end and a proximal end. At least one effecter is located in the distal end, being in a communication with the single actuating mechanism, located at the proximal portion of the tool. Preferably, the single actuating mechanism is accommodated at the proximal end, namely in or near the tool's handle.

It is also in the scope of the present invention to disclose the resectoscope as defined above, wherein said single actuating mechanism comprises at least one active handle. The handle is located at the vicinity of the proximal end and provides the effecter with both a linear motion and a rotational motion.

It is also in the scope of the present invention to disclose a resectoscope as defined above, comprising an effecter and single actuating mechanism itself comprising a rotation mechanism. The rotation mechanism of the resectoscope is selected in a non-limiting manner from rotating screw and fixed bolt, rotating bolt and fixed screw, a spiral track and travelling pin or any other means of producing rotational motion. The linear motion mechanism of the resectoscope is selected in a non-limiting manner from a sliding lock plate which pushes the rotational mechanism in a linear direction, a straight track and travelling pin or any other means of providing linear motion, such that an operator uses the active handle to produce both rotational and linear motion of the communicated effecter.

It is also in the scope of the present invention to disclose the resectoscope as defined in any of the above, wherein the linear mechanism is bolted to a springed arm; the extension of the springed arm is impeded by an active handle, by which the operator controls the extent of the linear motion, during the predetermined portion of the full range of the handle. When fully extended, the linear motion springed arm prevents any further movement of the linear movement mechanism and further wherein said rotational motion mechanism is bolted to the active handle such that its movement is controlled directly by the operator over the second portion of the full range of the active handle. It is acknowledged in this respect that the aforesaid resectoscope is alternatively characterized in the manner that the first motion is rotational and the second motion is linear. A resectoscope with a multiple motions sequence is also possible, e.g., a liner-rotational-liner motion, rotational-linear-rotational motion etc.

It is also in the scope of the present invention to disclose a resectoscope as defined in any of the above, wherein the electrical tension of the resector-loop is determined by the user such that a high tension is applied for resection and a lower tension is applied for cauterisation.

The rotational and linear mechanical phases are independent from the coagulation and resection electrical phases such that the cauterisation phase can be simultaneous with either any part of the rotational, any part of the linear phases or both and the resection phase can be simultaneous with either any part of the rotational, any part of the linear phases or both.

It is another object of the present invention to present an effective method of dual-action actuating. The method comprising steps selected inter alia from (a) interconnecting an effecter with a single actuating mechanism; and (b) activating said effecter in a sequence of rotational and linear maneuvers by means of said single actuating mechanism. A method of actuating a dual-action working tool is also provided, and comprising (a) providing at least one effecter; and (b) communicating said effecter with a single actuating mechanism. A method of actuating a dual-action endoscope is also provided, and comprising (a) providing at least one effecter located adjacent to a body portion to be treated in a sequence of rotational and linear maneuvers; and (b) communicating said effecter with a single actuating mechanism.

It is hence is the scope of the present invention to present an effective method of actuating a dual-action linear-rotational resectoscope. This method comprising steps selected inter alia from (a) obtaining a resection loop and loop shaft which are raised to high electrical tension for either resection or cauterisation; (b) obtaining an endoscope tube electrically insulated from the loop shaft; (c) obtaining an insulating sheath comprising an inner and outer sheath surrounding both the loop shaft and the endoscope tube; (d) allowing fluid to flow between said inner and outer sheathes; (e) controlling both linear and rotational motion of the resector-loop by means of a handle; (f) performing a plurality of resections by using either rotational motion of the resector-loop, linear motion of the resector-loop or any combination of the two; and (g) manoeuvring said resector-loop adjacent to a body portion to be treated in a sequence of rotational and linear maneuvers comprising a unified actuating module in communication with said effecter.

### BRIEF DESCRIPTION OF THE FIGURES

The objects and advantages of various embodiments of the invention will become apparent from the following description when read in conjunction with the accompanying drawings wherein

FIG. 1a schematically represents a side view of the resectoscope with the resection loop retracted within the sheaths, in this phase the active handle is in its most proximal position;

FIG. 1b schematically represents a side view that shows the resectoscope at the distal end of the linear action phase where the resection loop protrudes outmost from the sheaths into a position where it has freedom to rotate, during the linear phase the active handle moves along the first portion of its full range;

FIG. 1c schematically represents a side view of the resectoscope during the rotational action phase where the resection loop rotates around its axis, during this phase the active handle moves along the second portion of its full range;

FIG. 2 schematically represents an oblique view of the dual resectoscope with its outer sheaths removed such that the loop's shaft and the endoscope tube frame can be seen. Also apparent is the cable connecting the handset to the power supply unit;

FIG. 3 schematically represents the active handle of the dual resectoscope handset. Here the two hinged arms can be seen as well as the rotation screw and the loop's shaft release pin;

FIG. 4 schematically represents the dual movement mechanism showing the rotation movement mechanism comprising the rotation screw and bolt and the linear movement mechanism comprising the electrical connection sliding unit;

FIG. 5 schematically represents the loop's shaft release pin mechanism; and,

FIG. 6 schematically represents the electrical connector housed within the electrical connection sliding unit and adapted to provide a constant electrical connection whilst allowing angular limited free rotation of the rotation screw.

### DETAILED DESCRIPTION OF THE EMBODIMENTS

The following description is provided, alongside all chapters of the present invention, so as to enable any person skilled in the art to make use of said invention and sets forth the best modes contemplated by the inventor of carrying out this invention. Various modifications, however, will remain apparent to those skilled in the art, since the generic principles of the present invention have been defined specifically to provide a dual-action rotational and linear resectoscope.

The term ‘endoscope’ generally refers hereinafter to any medical device comprising a maneuverable effecter adapted to maneuver adjacent or inside a target tissue or organ. More specifically, the term refers to endoscopes comprising at least one effecter adapted to maneuver adjacent or inside a target tissue or organ inside a body cavity. Said endoscope may be selected in a non-limiting manner from resectoscope, laparoscope, cystoscope, arthroscope, lithotriptoscope, cysto-urethroscope, sinoscope, hysteroscope, colonoscope, neproscope, bronchoscope, choledochoscope, sigmoido-scope, arthroscope, utererscope, gastroscope or any other, either rigid or flexible, endoscopic surgical tool.

In a non-limiting manner, the term ‘resectoscope’ refers hereinafter to any surgical instrument for performing a resection without an opening or incision other than that made by the instrument.

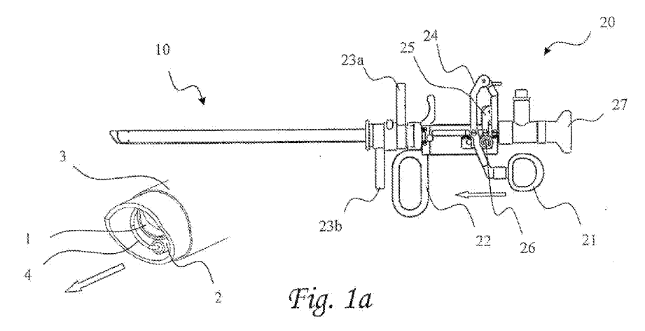

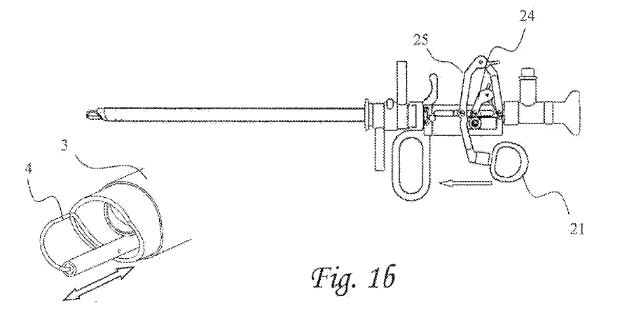

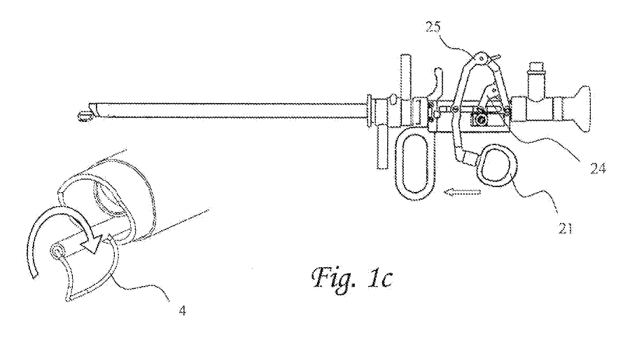

Reference is now made to FIG. 1a schematically representing a side view of the resectoscope in the retracted mode. The resectoscope comprises a shaft portion, 10, and a handset, 20. The shaft portion comprises the endoscope tube frame, 1, the loop's shaft, 2 and the outer sheaths, 3, in this mode the resection loop, 4 is retracted into the sheaths. The handset, 20, comprises the active handset, 21, the passive handset, 22, a fluid inlet, 23a, and outlet, 23b, the handle's movement hinged arm, 24, the linear motion hinged arm, 25, the electrical connector, 26, and the optical unit, 27, in this phase the active handle, 21, is in its most proximal position.

Reference is now made to FIG. 1b schematically representing a side view of the resectoscope at the distal end the linear action phase where the resection loop, 4, protrudes outmost from the outer sheaths, 3, into a position where it has freedom to rotate, during this phase the active handle, 21, moves along the first portion of its full range.

Reference is now made to FIG. 1c schematically representing a side view of the resectoscope during the rotational action phase where the resection loop, 4, rotates around its axis, during this phase the active handle, 21, moves along the second portion of its full range.

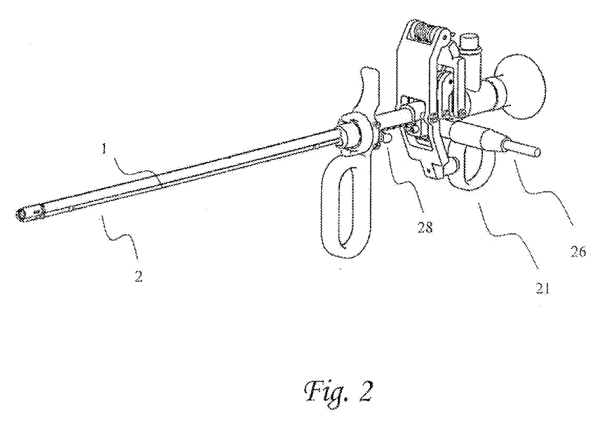

Reference is now made to FIG. 2 schematically representing an oblique view of the dual resectoscope with its outer sheaths removed such that the loop's shaft, 2, and the endoscope tube frame, 1, can be seen. Also apparent is the cable connecting the handset to the power supply unit (not shown) and the loop's shaft release pin, 28.

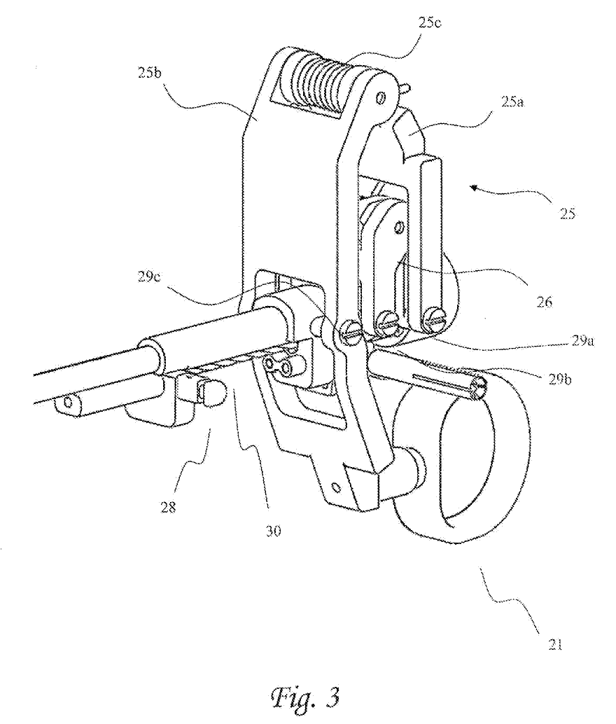

Reference is now made to FIG. 3 schematically representing the active handle, 21, of the dual resectoscope handset. Here the two hinged arms, 25 and 26, can be seen as well as the rotation screw, 30, and the loop's shaft release pin, 28. The handle's movement hinged arm, 25, comprises a rear segment, 25a, a fore segment, 25b, and a spring, 25c. The rear segment of the handle's movement hinged arm, 25a, is hinged to the handset at a fixed axis, 29a, at the proximal end of the handset, a fore segment, 25b, which is hinged to the rear segment, at the spring, 25c, and hinged to the handset at a sliding axis, 29c. The linear movement hinged arm, 26, comprises a rear segment, a fore segment and a spring. The rear segment of the handle's movement hinged arm is hinged to the handset at the fixed axis, 29a, at the proximal end of the handset, the fore segment which is hinged to the rear segment at a springed hinge (not shown) and hinged to the handset at a sliding axis, 29b.

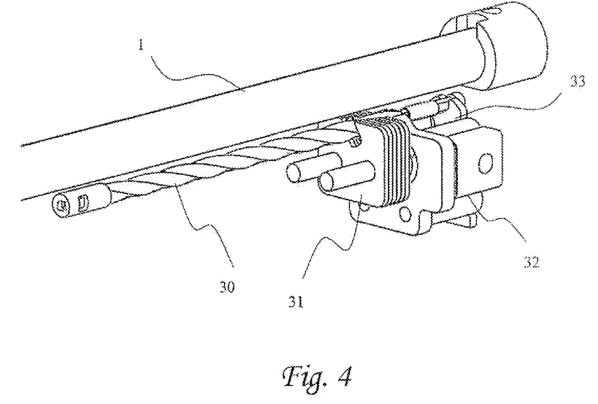

Reference is now made to FIG. 4 schematically representing the dual movement mechanism showing the rotation movement mechanism comprising the rotation screw, 30, and bolt, 31, as well as the linear movement mechanism comprising the electrical connection sliding unit, 32, and linear movement lock plate, 33.

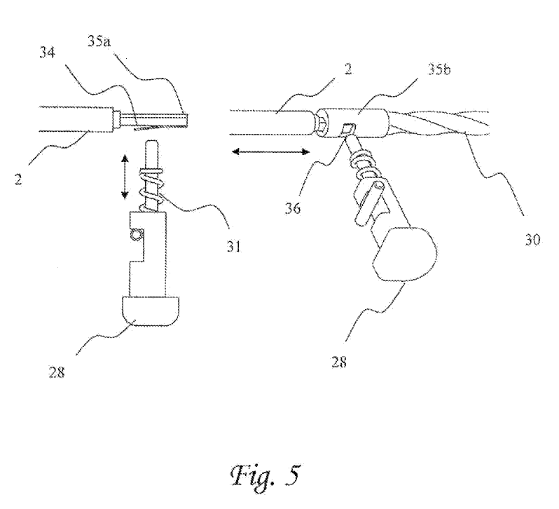

Reference is now made to FIG. 5 schematically representing the loop's shaft release pin mechanism comprising the loop's shaft release pin, 28, and spring, 31. The male-connector, 35a, of the loop's shaft, 2, has a conducting catch, 34, when the connector is plugged into the female-connector, 35b, of the rotation screw, 30, the catch rises into the female-connector window, 36, preventing the male-connector from disconnecting and ensuring that the two are in good electrical contact. In order to disconnect the loop's shaft from the rotation screw, the release pin, 28, is pressed down upon the conducting catch, 34, and the male-connector is released to slide out of the female-connector.

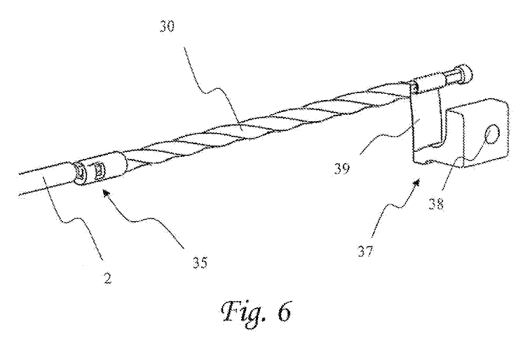

Reference is now made to FIG. 6 schematically representing the electrical connector, 37, housed within the electrical connection sliding unit and adapted to provide a constant electrical connection whilst allowing angular limited free rotation of the rotation screw, 30. The electrical cable (not shown) passes through the circular hole, 38, making contact with part 37, which is welded to a thin strip of flexible and highly conductive material, 39, such as gold or silver which is attached to the rotation screw, 30, with sufficient freedom of angular motion so as to allow the rotation screw to rotate without breaking the strip. Electrical connection between the rotation screw, 30, and the loop's shaft, 2, is provided by the connector, 35.

1. A dual-action actuating system, interconnecting an effecter with a single actuating mechanism activating said effecter in a sequence of rotational and linear maneuvers, said maneuvers are selected from a group consisting of linear motion followed by rotational motion, rotational motion followed by linear motion, only rotational motion and only linear motion.

2. A working tool comprising the dual-action system as defined in claim 1; said tool having a main longitudinal axis having a distal end and a proximal end; at least one effecter is located in the distal end, being in a communication with the single actuating mechanism, located at the proximal portion of the tool.

3. The working tool according to claim 2, wherein the single actuating mechanism is accommodated at the proximal end, namely in or near the tool's handle.

4. A working tool according to claim 2, wherein said single actuating mechanism comprises at least one active handle, said handle is located at the vicinity of the proximal end and provides the effecter with both a linear motion and a rotational motion.

5. A working tool according to claim 4, comprising an effecter and single actuating mechanism itself comprising a rotation mechanism selected from rotating screw and fixed bolt, rotating bolt and fixed screw, a spiral track and travelling pin or any other means of producing rotational motion; and a linear motion mechanism selected from a sliding lock plate which pushes the rotational mechanism in a linear direction, a straight track and travelling pin or any other means of providing linear motion, such that an operator uses the active handle to produce both rotational and linear motion of the communicated effecter.

6. A working tool according to claim 4, wherein said linear mechanism is bolted to a springed arm; the extension of the springed arm is impeded by an active handle, by which the operator controls the extent of the linear motion, during the predetermined portion of the full range of the handle; when fully extended, the linear motion springed arm prevents any further movement of the linear movement mechanism and further wherein said rotational motion mechanism is bolted to the active handle such that its movement is controlled directly by the operator over the second portion of the full range of the active handle.

7. An endoscope having a dual-action actuating mechanism according to claim 1 useful for manoeuvring at least one effecter located adjacent to a body portion to be treated in a sequence of rotational and linear maneuvers, said maneuvers are selected from a group consisting of linear motion followed by rotational motion, rotational motion followed by linear motion, only rotational motion and only linear motion; said endoscope comprising a unified actuating module in communication with said effecter.

8. An endoscope comprising the dual-action system as defined in claim 7; said tool having a main longitudinal axis having a distal end and a proximal end; at least one effecter is located in the distal end, being in a communication with the single actuating mechanism, located at the proximal portion of the tool.

9. The endoscope according to claim 8, wherein the single actuating mechanism is accommodated at the proximal end, namely in or near the tool's handle.

10. An endoscope according to claim 8, wherein said single actuating mechanism comprises at least one active handle, said handle is located at the vicinity of the proximal end and provides the effecter with both a linear motion and a rotational motion.

11. A endoscope according to claim 10, comprising an effecter and single actuating mechanism itself comprising a rotation mechanism selected from rotating screw and fixed bolt, rotating bolt and fixed screw, a spiral track and travelling pin or any other means of producing rotational motion; and a linear motion mechanism selected from a sliding lock plate which pushes the rotational mechanism in a linear direction, a straight track and travelling pin or any other means of providing linear motion, such that an operator uses the active handle to produce both rotational and linear motion of the communicated effecter.

12. A endoscope according to claim 10, wherein said linear mechanism is bolted to a springed arm; the extension of the springed arm is impeded by an active handle, by which the operator controls the extent of the linear motion, during the predetermined portion of the full range of the handle; when fully extended, the linear motion springed arm prevents any further movement of the linear movement mechanism and further wherein said rotational motion mechanism is bolted to the active handle such that its movement is controlled directly by the operator over the second portion of the full range of the active handle.

13. The endoscope according to claim 7, wherein said endoscope is selected from resectoscope, laparoscope, cystoscope, arthroscope, lithotriptoscope, cysto-urethroscope, sinoscope, hysteroscope, colonoscope, neproscope, bronchoscope, choledochoscope, sigmoido-scope, arthroscope, utererscope, gastroscope or any other, either rigid or flexible, endoscopic surgical tool.

14. A dual action linear-rotational resectoscope comprising;
a. a resection loop and loop shaft which are raised to high electrical tension for either resection or cauterisation;
b. an endoscope tube electrically insulated from the loop shaft;
c. an insulating sheath comprising an inner and outer sheath between which fluid is free to flow, surrounding both the loop shaft and the endoscope tube;
d. a handle by which an operator controls both linear and rotational motion of the resector-loop such that the operator performs a resection by using either rotational motion of the resector-loop, linear motion of the resector-loop or any combination of the two; and
e. a dual-action actuating mechanism useful for manoeuvring said resector-loop adjacent to a body portion to be treated in a sequence of rotational and linear maneuvers; said maneuvers are selected from a group consisting of linear motion followed by rotational motion, rotational motion followed by linear motion, only rotational motion and only linear motion; comprising a unified actuating module in communication with said effecter.

15. A resectoscope comprising the dual-action system as defined in claim 14; said tool having a main longitudinal axis having a distal end and a proximal end; at least one effecter is located in the distal end, being in a communication with the single actuating mechanism, located at the proximal portion of the tool.

16. The resectoscope according to claim 15, wherein the single actuating mechanism is accommodated at the proximal end, namely in or near the tool's handle.

17. The resectoscope according to claim 15, wherein said single actuating mechanism comprises at least one active handle, said handle is located at the vicinity of the proximal end and provides the effecter with both a linear motion and a rotational motion.

18. A resectoscope according to claim 17, comprising an effecter and single actuating mechanism itself comprising a rotation mechanism selected from rotating screw and fixed bolt, rotating bolt and fixed screw, a spiral track and travelling pin or any other means of producing rotational motion; and a linear motion mechanism selected from a sliding lock plate which pushes the rotational mechanism in a linear direction, a straight track and travelling pin or any other means of providing linear motion, such that an operator uses the active handle to produce both rotational and linear motion of the communicated effecter.

19. A resectoscope according to claim 17, wherein said linear mechanism is bolted to a springed arm; the extension of the springed arm is impeded by an active handle, by which the operator controls the extent of the linear motion, during the predetermined portion of the full range of the handle; when fully extended, the linear motion springed arm prevents any further movement of the linear movement mechanism and further wherein said rotational motion mechanism is bolted to the active handle such that its movement is controlled directly by the operator over the second portion of the full range of the active handle.

20. The resectoscope according to claim 17, wherein the electrical tension of the resector-loop is determined by the user such that a high tension is applied for resection and a lower tension is applied for cauterisation.

21. The resectoscope according to claim 20 wherein the rotational and linear mechanical phases are independent from the coagulation and resection electrical phases such that the cauterisation phase can be simultaneous with either any part of the rotational, any part of the linear phases or both and the resection phase can be simultaneous with either any part of the rotational, any part of the linear phases or both.

22. A method of actuating a dual-action endoscope comprising; providing at least one effecter located adjacent to a body portion to be treated in a sequence of rotational and linear maneuvers; said maneuvers are selected from a group consisting of linear motion followed by rotational motion, rotational motion followed by linear motion, only rotational motion and only linear motion; and, communicating said effecter with a single actuating mechanism.

23. A method of actuating a dual-action linear-rotational resectoscope comprising;
a. obtaining a resection loop and loop shaft which are raised to high electrical tension for either resection or cauterisation;
b. obtaining an endoscope tube electrically insulated from the loop shaft;
c. obtaining an insulating sheath comprising an inner and outer sheath surrounding both the loop shaft and the endoscope tube;
d. allowing fluid to flow between said inner and outer sheathes;
e. controlling both linear and rotational motion of the resector-loop by means of a handle;
f. performing a plurality of resections by using either rotational motion of the resector-loop, linear motion of the resector-loop or any combination of the two; and
g. manoeuvring said resector-loop adjacent to a body portion to be treated in a sequence of rotational and linear maneuvers comprising a unified actuating module in communication with said effecter.
